SEQUENCE LISTING (25 sequences)

SEQ ID NO: 1 - DNA, 20 bp, Artificial sequence
  Synthetic
gaaccaacactcgatcaccg

SEQ ID NO: 2 - DNA, 20 bp, Artificial sequence
  Synthetic
gccccggggataccgcctga

SEQ ID NO: 3 - DNA, 20 bp, Artificial sequence
  Synthetic
ggtgcgcaccgtccttcgcc

SEQ ID NO: 4 - DNA, 20 bp, Artificial sequence
  Synthetic
ctcagtggcaagttcagcgt

SEQ ID NO: 5 - DNA, 20 bp, Artificial sequence
  Synthetic
tcacgtcatccagcagagaa

SEQ ID NO: 6 - DNA, 20 bp, Artificial sequence
  Synthetic
cacagcccaagatagttaag

SEQ ID NO: 7 - DNA, 27 bp, Artificial sequence
  Synthetic
agcgccatgaagtccctgtctctgctc

SEQ ID NO: 8 - DNA, 24 bp, Artificial sequence
  Synthetic
ggcacgaagcggctcatctcgcag

SEQ ID NO: 9 - DNA, 26 bp, Artificial sequence
  Synthetic
gcaaatccaggactttaactctcacc

SEQ ID NO: 10 - DNA, 25 bp, Artificial sequence
  Synthetic
gagatggagtttcgctcttgttgcc

SEQ ID NO: 11 - DNA, 20 bp, Artificial sequence
  Synthetic
gggaaggtggaagctcattt

SEQ ID NO: 12 - DNA, 23 bp, Artificial sequence
  Synthetic
ctagaggaagccagtaggtaaga

SEQ ID NO: 13 - protein, 5 aa, Artifical sequence
KKKAE

SEQ ID NO: 14 - protein, 504 aa, Homo sapien
MKSLSLLLAVALGLATAVSAGPAVIECWFVEDASGKGLAKRPGALLLRQGPGEPPPRPDLDPELYLSVHDPAGALQAAFRRYPRGAPAPHCEMSRFVPLPASAKWASGLTPAQNCPRALDGAWLMVSISSPVLSLSSLLRPQPEPQQEPVLITMATVVLTVLTHTPAPRVRLGQDALLDLSFAYMPPTSEAASSLAPGPPPFGLEWRRQHLGKGHLLLAATPGLNGQMPAAQEGAVAFAAWDDDEPWGPWTGNGTFWLPTVQPFQEGTYLATIHLPYLQGQVTLELAVYKPPKVSLMPATLARAAPGEAPPELLCLVSHFYPSGGLEVEWELRGGPGGRSQKAEGQRWLSALRHHSDGSVSLSGHLQPPPVTTEQHGARYACRIHHPSLPASGRSAEVTLEVAGLSGPSLEDSVGLFLSAFLLLGLFKALGWAAVYLSTCKDSKKVQCSTSLYLSLVTLSPHPISKPMEGGCWCGRQNLGLEFTLIWVKTWHYILTVGLFEHAT

SEQ ID NO: 15 - protein, 448 aa, Homo sapien
MKSLSLLLAVALGLATAVSAGPAVIECWFVEDASGKGLAKRPGALLLRQGPGEPPPRPDLDPELYLSVHDPAGALQAAFRRYPRGAPAPHCEMSRFVPLPASAKWASGLTPAQNCPRALDGAWLMVSISSPVLSLSSLLRPQPEPQQEPVLITMATVVLTVLTHTPAPRVRLGQDALLDLSFAYMPPTSEAASSLAPGPPPFGLEWRRQHLGKGHLLLAATPGLNGQMPAAQEGAVAFAAWDDDEPWGPWTGNGTFWLPRVQPFQEGTYLATIHLPYLQGQVTLELAVYKPPKVSLMPATLARAAPGEAPPELLCLVSHFYPSGGLEVEWELRGGPGGRSQKAEGQRWLSALRHHSDGSVSLSGHLQPPPVTTEQHGARYACRIHHPSLPASGRSAEVTLEVAGLSGPSLEDSVGLFLSAFLLLGLFKALGWAAVYLSTCKDSKKKAE

SEQ ID NO: 16 - protein, 466 aa, Homo sapien
MKSLSLLLAVALGLATAVSAGPAVIECWFVEDASGKGLAKRPGALLLRQGPGEPPPRPDLDPELYLSVHDPAGALQAAFRRYPRGAPAPHCEMSRFVPLPASAKWASGLTPAQNCPRALDGAWLMVSISSPVLSLSSLLRPQPEPQQEPVLITMATVEMGFHHVGQAALELLTSVVLTVLTHTPAPRVRLGQDALLDLSFAYMPPTSEAASSLAPGPPPFGLEWRRQHLGKGHLLLAATPGLNGQMPAAQEGAVAFAAWDDDEPWGPWTGNGTFWLPTVQPFQEGTYLATIHLPYLQGQVTLELAVYKPPKVSLMPATLARAAPGEAPPELLCLVSHFYPSGGLEVEWELRGGPGGRSQKAEGQRWLSALRHHSDGSVSLSGHLQPPPVTTEQHGARYACRIHHPSLPASGRSAEVTLEVAGLSGPSLEDSVGLFLSAFLLLGLFKALGWAAVYLSTCKDSKKKAE

SEQ ID NO: 17 - protein, 466 aa, Homo sapien
MKSLSLLLAVALGLATAVSAGPAVIECWFVEDASGKGLAKRPGALLLRQGPGEPPPRPDLDPELYLSVHDPAGALQAAFRRYPRGAPAPHCEMSRFVPLPASAKWASGLTPAQNCPRALDGAWLMVSISSPVLSLSSLLRPQPEPQQEPVLITMATVEMGFHHVGQAALELLTSVVLTVLTHTPAPRVRLGQDALLDLSFAYMPPTSEAASSLAPGPPPFGLEWRRQHLGKGHLLLAATPGLNGQMPAAQEGAVAFAAWDDDEPWGPWTGNGTFWLPRVQPFQEGTYLATIHLPYLQGQVTLELAVYKPPKVSLMPATLARAAPGEAPPELLCLVSHFYPSGGLEVEWELRGGPGGRSQKAEGQRWLSALRHHSDGSVSLSGHLQPPPVTTEQHGARYACRIHHPSLPASGRSAEVTLEVAGLSGPSLEDSVGLFLSAFLLLGLFKALGWAAVYLSTCKDSKKKAE

SEQ ID NO: 18 - protein, 448 aa, Homo sapien
MKSLSLLLAVALGLATAVSAGPAVIECWFVEDASGKGLAKRPGALLLRQGPGEPPPRPDLDPELYLSVHDPAGALQAAFRRYPRGAPAPHCEMSRFVPLPASAKWASGLTPAQNCPRALDGAWLMVSISSPVLSLSSLLRPQPEPQQEPVLITMATVVLTVLTHTPAPRVRLGQDALLDLSFAYMPPTSEAASSLAPGPPPFGLEWRRQHLGKGHLLLAATPGLNGQMPAAQEGAVAFAAWDDDEPWGPWTGNGTFWLPRVQPFQEGTYLATIHLPYLQGQVTLELAVYKPPKVSLMPATLARAAPGEAPPELLCLVSHFYPSGGLEVEWELRGGPGGRSQKAEGQRWLSALRHHSDGSVSLSGHLQPPPVTTEQHGARYACRIHHPSLPASGRSAEVTLEVAGLSGPSLEDSVGLFLSAFLLLGLFKALGWAAVYLSTCKDSKKHAT

SEQ ID NO: 19 - protein, 361 aa, Homo sapien
MKSLSLLLAVALGLATAVSAGPAVIECWFVEDASGKGLAKRPGALLLRQGPGEPPPRPDLDPELYLSVHVVLTVLTHTPAPRVRLGQDALLDLSFAYMPPTSEAASSLAPGPPPFGLEWRRQHLGKGHLLLAATPGLNGQMPAAQEGAVAFAAWDDDEPWGPWTGNGTFWLPTVQPFQEGTYLATIHLPYLQGQVTLELAVYKPPKVSLMPATLARAAPGEAPPELLCLVSHFYPSGGLEVEWELRGGPGGRSQKAEGQRWLSALRHHSDGSVSLSGHLQPPPVTTEQHGARYACRIHHPSLPASGRSAEVTLEVAGLSGPSLEDSVGLFLSAFLLLGLFKALGWAAVYLSTCKDSKKKAE

SEQ ID NO: 20 - protein, 192 aa, Homo sapien
MKSLSLLLAVALDPAGALQAAFRRYPRGAPAPHCEMSRFVPLPASAKWASGLTPAQNCPRALDGAWLMVSISSPVLSLSSLLRPQPEPQQEPVLITMATVVLTVLTHTPAPRVRLGQDALLDLSFAYMPPTSEAASSLAPGPPPFGLEWRRQHLGKGHLLLAATPGLNGQMPAAQEGAVAFAAWDDDEPWGP

SEQ ID NO: 21 - protein, 156 aa, Homo sapien
MKSLSLLLAVALGLATAVSAGPAVIECWFVEDASGKGLAKRPGALLLRQGPGEPPPRPDLDPELYLSVHDPAGALQAAFRRYPRGAPAPHCEMSRFVPLPASAKWASGLTPAQNCPRALDGAWLMVSISSMGHAMPVEFTIPACLPRGAALRSPWR

SEQ ID NO: 22 - protein, 202 aa, Homo sapien
MKSLSLLLAVALGLATAVSAGPAVIECWFVEDASGKGLAKRPGALLLRQGPGEPPPRPDLDPELYLSVHVVLTVLTHTPAPRVRLGQDALLDLSFAYMPPTSEAASSLAPGPPPFGLEWRRQHLGKGHLLLAATPGLNGQMPAAQEGAVAFAAWDDDEPWGPWTGNGTFWLPRVQPFQEGTYLATIHLPYLQGQVTLELAVY

SEQ ID NO: 23 - protein, 202 aa, Homo sapien
MKSLSLLLAVALGLATAVSAGPAVIECWFVEDASGKGLAKRPGALLLRQGPGEPPPRPDLDPELYLSVHVVLTVLTHTPAPRVRLGQDALLDLSFAYMPPTSEAASSLAPGPPPFGLEWRRQHLGKGHLLLAATPGLNGQMPAAQEGAVAFAAWDDDEPWGPWTGNGTFWLPTVQPFQEGTYLATIHLPYLQGQVTLELAVY

SEQ ID NO: 24 - protein, 468 aa, Homo sapien
MGTQEGWCLLLCLALSGAAETKPHPAEGQWRAVDVVLDCFLAKDGAHRGALASSEDRARASLVLKQVPVLDDGSLEDFTDFQGGTLAQDDPPIIFEASVDLVQIPQAEALLHADCSGKEVTCEISRYFLQMTETTVKTAAWFMANMQVSGGGPSISLVMKTPRVTKNEALWHPTLNLPLSPQGTVRTAVEFQVMTQTQSLSFLLGSSASLDCGFSMAPGLDLISVEWRLQHKGRGQLVYSWTAGQGQAVRKGATLEPAQLGMARDASLTLPGLTIQDEGTYICQITTSLYRAQQIIQLNIQASPKVRLSLANEALLPTLICDIAGYYPLDVVVTWTREELGGSPAQVSGASFSSLRQSVAGTYSISSSLTAEPGSAGATYTCQVTHISLEEPLGASTQVVPPERRTALGVIFASSLFLLALMFLGLQRRQAPTGLGLLQAERWETTSCADTQSSHLHEDRTARVSQPS

SEQ ID NO: 25 - protein, 185 aa, Homo sapien
MGTQEGWCLLLCLALSGAAETMDLVQIPQAEALLHADCSGKEVTCEISRYFLQMTETTVKTAAWFMANMQVSGGGPSISLVMKTPRVTKNEALWHPTLNLPLSPQGTVRTAVEFQVMTQTQSLSFLLGSSASLDCGFSMAPGLDLISVEWRLQHKGRGRGDLHLPDHHLSVPSSADHPAQHPSFP

(19) United States
(12) Patent Application Publication
(10) Pub. No.: US 2023/0123128 A1
(43) Pub. Date: Apr. 20, 2023

(54) TARGETING TAPASIN AND TAP COMPLEX TO IMPROVE CELLULAR IMMUNE-COMPATIBILITY

(71) Applicant: PROGENITOR LIFE SCIENCES, Salt Lake City, UT (US)

(72) Inventors: Timothy E. GRAHAM, Sandy, UT (US); Patricia Rosa DE ARAUJO, Centerville, UT (US)

(21) Appl. No.: 17/914,074

(22) PCT Filed: Apr. 1, 2021
(86) PCT No.: PCT/US2021/025311

§ 371 Date: Sep. 23, 2022

Related U.S. Application Data
(60) Provisional application No. 63/004,923, filed on Apr. 3, 2020.

(51) Int. Cl. C12N 15/09; C12N 5/071; C07K 14/74

(52) U.S. Cl. CPC C12N 15/09; C12N 5/0602; C07K 14/70539; C12N 2510/00; C12N 2310/20

(57) ABSTRACT

Cells, compositions, and methods for producing and transplanting cells which exhibit reduced antigen presentation by the major histocompatibility complex type I (MHC-I) by decreasing or eliminating activity of a tapasin protein or homolog thereof are provided.

This patent application claims the benefit of priority from U.S. Provisional Application Ser. No. 63/004,923 filed Apr. 3, 2020, teachings of which are herein incorporated by reference in their entirety.

### FIELD

This invention relates to cells and methods for producing and transplanting cells which exhibit reduced antigen presentation by the major histocompatibility complex type I (MHC-I) by decreasing or eliminating activity of a tapasin protein or a homolog thereof.

### BACKGROUND

There are over 200 distinct loci on human Chromosome 6 that regulate human histocompatibility, including 20 distinct human leukocyte antigen (HLA) genes encoding key parts of the major histocompatibility complexes, MHC-I and MHC-II. MHC-I is present on the cell surface of all nucleated cells in the body and functions to display peptide fragments of non-self proteins from within the cells to cytotoxic T cells (CTLs), thereby triggering immediate immune responses. Because MHC-I molecules present peptides derived from cytosolic proteins degraded by the proteasome, the pathway of MHC-I presentation is often called cytosolic or ‘endogenous’. Processed peptides are imported into the endoplasmic reticulum, assembled with nascent MHC-I complexes, and then inserted into the plasma membrane for external display. A normal cell will display peptides from normal cellular protein turnover on its class I MHC, and CTLs will not be activated in response to them due to central and peripheral tolerance mechanisms. When a cell expresses foreign proteins, such as after viral infections, a fraction of the class I MHC will display (present) these peptides on the cell surface. Consequently, T lymphocytes (T cells) specific for the MHC:peptide complex will recognize and kill the presenting cells.

MHC-I molecules are heterodimers of two polypeptide chains, an α chain and β2-microglobulin (B2M). The α chain is comprised of three distinct domains (α1, α2, and α3), and the α and β chains are linked non-covalently via interaction of B2M and the α3 domain. The α3 domain is plasma membrane-spanning and interacts with the CD8 co-receptor of T-cells. The α3-CD8 interaction holds the MHC I molecule in place while the T cell receptor (TCR) on the surface of the cytotoxic T cell binds its α1-α2 heterodimer ligand and checks the coupled peptide for antigenicity. The α1 and α2 domains fold to make up a groove for peptides to bind. MHC class I molecules bind peptides that are 8-10 amino acids in length. The a chain is comprised of proteins drawn from six different genetic HLA loci, whereas there is only one β chain, B2M, shared across all forms of MHC-I.

MHC-I is expressed by all cells and plays an important role in the process of allorecognition, wherein donor cells are detected and destroyed by the immune system of non-HLA-matched recipients after transplantation. A pathway of direct allorecognition exists whereby recipient T cells recognize donor MHC-I:peptide complexes displayed on the surface of transplanted cells. While there can be direct recognition of non-HLA matched MHC-I by recipient T cells, recipient allorecognition also depends on presentation of processed MHC-I peptides to recipient T cells via MHC-I of the donor cells. In transplant medicine, there are hundreds of different HLA-types. Thus, the requirement for one-on-one matching of donor and recipient by HLA-type in order to prevent rejection due to allorecognition presents a major limitation for use of transplanted cells or tissues as medical therapies.

In an effort to improve donor-recipient immune compatibility, several groups have proposed engineering immune cells with genetic knockout (KO) of B2M to eliminate MHC-I. However, MHC-I also functions as an important inhibitory ligand for natural killer cells (NKCs), such that loss of cell surface MHC-I expression due to B2M KO induces rapid destruction of cells by NKCs. In fact, loss of MHC-I expression is a more potent inducer of NKC-mediated destruction than MHC-I-mismatch. This well-recognized phenomenon has been called the “missing self” response. Newly assembled major histocompatibility complex (MHC) class I molecules, together with the endoplasmic reticulum (ER) chaperone calreticulin, interact with the transporter associated with antigen processing (TAP1) through a molecule called tapasin (also known as TAP-binding protein or TAPBP, Sadasivan et al., 1996). By molecular cloning of tapasin, Ortmann et al. (1997) found it to be a type I transmembrane glycoprotein encoded by an MHC-linked gene. The mature protein has 428 amino acids with a single N-linked glycosylation site at position 233. It is a member of the immunoglobulin superfamily with a probable cytoplasmic ER retention signal. Up to 4 MHC-I/tapasin complexes were found to bind to each TAP molecule in Daudi and L001 cells. Expression of tapasin in a negative mutant human cell line restored class I/TAP association and normal class I cell surface expression. Tapasin expression also corrected the defective recognition of virus-infected cells of the same line by class I-restricted cytotoxic T cells, thus establishing a critical functional role for tapasin in MHC-I-restricted antigen processing. Herberg et al. (1998) identified an EST encoding the mouse tapasin homolog.

Tapasin is required for efficient peptide loading of the MHC-I TAP complex. Up to four complexes of MHC-I and this protein may be bound to a single TAP molecule. This protein contains a C-terminal double-lysine motif (KKKAE; SEQ ID NO:13) known to maintain membrane proteins in the endoplasmic reticulum. This gene lies within the major histocompatibility complex on chromosome 6. Alternative splicing results in three transcript variants encoding different isoforms.

Mayer and Klein (2001) proposed that tapasin is in reality an MHC-I molecule with a different function from that currently executed by conventional class I molecules. They based this proposal on the amino acid sequence similarity between tapasin and conventional class I molecules, on similarity of predicted tertiary structure and domain organization of the molecules, on similarity of exon/intron organization of the encoding genes, and on the mapping of the class IA and tapasin genes into the same chromosomal region in all jawed vertebrates that had been tested to that time (Michalova et al., 2000).

Yabe et al. (2002) disclosed that HLA class I expression depends on the formation of a peptide-loading complex composed of class I heavy chain; B2M; TAP; and tapasin, which links TAP to the heavy chain. Using purified proteins, Rizvi and Raghavan (2006) demonstrated direct binding of tapasin to peptide-deficient MHC-I molecules at physiologic temperatures. Tapasin also bound mouse M10.5, a pheromone receptor-associated protein with a class I-like fold. Class I-tapasin complexes containing B2M assembled more rapidly with peptides than complexes lacking B2M. Peptide loading of class I inhibited class I-tapasin binding, whereas peptide depletion enhanced binding.

Herberg et al. (1998) determined that the coding sequence of the tapasin gene contains 8 exons, spanning approximately 12 kb. Teng et al. (2002) noted the presence of a C-terminal KKxx ER retention motif in the tapasin gene. The promoter region of tapasin contains gamma-interferon (IFNG) induction elements, but no TATA or CAATT boxes.

Ortmann et al. (1997) mapped the tapasin gene (TAPBP) to 6p21.3 by analysis of somatic cell hybrids containing chromosome 6 fragments and fluorescence in situ hybridization. Herber et al. (1998) localized the tapasin gene to a position between the HSET (603763) and HKE1.5 genes and within 500 kb of the TAP I and TAP 2 (170261) genes. By inclusion within mapped clones, they determined that mouse tapasin maps to chromosome 17 in a region showing homology of synteny with human chromosome 6p21.3.

Mutations in the TAP2 gene (e.g., 170261.0004) result in an HLA class I deficiency called type I bare lymphocyte syndrome (BLS). Yabe et al. (2002) described a 54-year-old woman with type I BLS who did not exhibit apparent TAP abnormalities but who had a tapasin defect. The gene encoding tapasin had a 7.4-kb deletion between introns 3 and 7 cause by an Alu repeat-mediated unequal homologous recombination (601962.0001). No tapasin polypeptide was detected in the subject's cells. The cell surface class I expression level in tapasin-deficient cells was markedly reduced but the reduction was not as profound as in TAP-deficient cells. Thus, tapasin deficiency is another cause of type I BLS.

Mice with whole body (germline) targeted knockout of tapasin (Tapasin KO mice) exhibit impaired folding, processing, and presentation of HLA peptides on MHC-I. Humans with inborn deficiency of tapasin similarly exhibit impaired cytotoxic T cell responses and increased susceptibility to infectious disease and neoplasia. In many cases, tapasin function can be manipulated such that MBC-I itself continues to be expressed (albeit at lower levels) on the surface of tapasin KO cells such that NKC-mediated ‘missing-self’ responses are not elicited. In addition, tapasin knockout does not affect the half-life of MHC-I molecules. Because tapasin expression is absent from conception and throughout development, NKCs of Tapasin KO mice develop a unique tolerance to missing-self; as a result, Tapasin KO mice NKCs transferred heterologously to blood of unmatched wildtype recipient mice do not themselves exhibit normal cytotoxic activity. See Garbi et al. Nat Immunol. 2000 September; 1(3):234-8. PubMed PMID: 10973281 and Boulanger et al. J Immunol. 2010 Jan. 1; 184(1):73-83. doi:10.4049/jimmunol.0803489. Epub 2009 Nov. 30. PubMed PMID: 19949070.

TAP Binding Protein-Like (Tapbpl;tapasin-like) is another component of the antigen processing and presentation pathway, which binds to MHC class I coupled with beta2-microglobulin/B2M. Association between TAPBPL and MHC class I occurs in the absence of a functional peptide-loading complex (PLC). See Boyle et al. Proc. Natl. Acad. Sci. U.S.A. 2013 110:3465-3470 and Morozov et al. Proc. Natl. Acad. Sci. U.S.A. 2016 113:E1006-E1015. TAPBPL is a member of the Ig superfamily that is localized on chromosome 12p13.3, a region somewhat paralogous to the MHC. See https: with the extension ncbi.nlm.nih.gov/gene/55080 of the world wide web.

New methods are needed to reduce CTL-mediated destruction of mismatched cells without triggering the missing-self response.

### SUMMARY

An aspect of the present invention relates to a method for producing a cell which exhibits reduced antigen presentation by the major histocompatibility complex type I (MHC-I). The method comprises decreasing or eliminating activity of a tapasin protein or homolog thereof in one or more cells to be transplanted into an allogeneic recipient.

Another aspect of the present invention relates to a method for transplanting one or more cells into an allogenic recipient. The method comprises preparing one or more cells in which activity of an endogenous tapasin protein is decreased or eliminated and transplanting the one or more cells into the allogeneic recipient.

In one nonlimiting embodiment of these methods, activity of the tapasin protein or a homolog thereof is decreased or eliminated by inhibiting expression of a nucleic acid sequence encoding the tapasin protein or homolog thereof.

In one nonlimiting embodiment of these methods, activity of the tapasin protein or homolog thereof is decreased or eliminated by modifying the one or more cells to express a nucleic acid sequence encoding a dominant-negative mutant of tapasin or the homolog thereof.

In one nonlimiting embodiment of these methods, activity of the tapasin protein or homolog thereof is decreased or eliminated by modifying the one or more cells to constitutively express a viral immunoevasin protein.

Another aspect of the present invention relates to a cell comprising a cellular genome which lacks at least a portion of an endogenous tapasin gene or homolog thereof so that expression of the tapasin protein or homolog thereof in the cell is decreased or eliminated.

Another aspect of the present invention relates to a cell comprising a cellular genome modified to express a nucleic acid sequence encoding a dominant-negative mutant of tapasin or homolog thereof and/or a nucleic acid sequence encoding viral immunoevasin protein.

Another aspect of the present invention relates to a nucleic acid sequence comprising a 5′ homology arm complementary to a tapasin gene or portion thereof or homolog thereof, a 3′ homology arm complementary to a tapasin gene or portion thereof or homolog thereof, and at least one exogenous polynucleotide. In one nonlimiting embodiment, the 5′ homology arm and the 3′ homology arm are complementary to different parts of a tapasin gene region of a host cell.

Another aspect of the present invention relates to vectors and host cells comprising a vector comprising the nucleic acid sequences comprising a 5′ homology arm complementary to a tapasin gene or portion thereof or homolog thereof, a 3′ homology arm complementary to a tapasin gene or portion thereof or homolog thereof, and at least one exogenous polynucleotide.

Yet another aspect of the present invention relates to a method for inhibiting tapasin expression or function in a host cell via incorporation of a nucleic acid sequence or vector comprising a 5′ homology arm complementary to a tapasin gene or portion thereof or homolog thereof, a 3′ homology arm complementary to a tapasin gene or portion thereof or homolog thereof, and at least one exogenous polynucleotide into the host cell.

### BRIEF SUMMARY OF THE FIGURES

FIG. 1 is a schematic summarizing the role of tapasin and tapasin-like proteins in the antigen presentation pathway as described by Hateren et al. F1000 Research 2017 6:158.

FIGS. 2A through 2C show genomic PCRs demonstrating the partial deletion of specific genes in a line of induced pluripotent stem cells (iPSCs): tapasin (FIG. 2A), tapasin and tapasin-like (FIG. 2B), and β2-microglobulin (FIG. 2C). In all cases, clones were derived from single cells obtained by limiting dilution. Knockout clones show the presence of a smaller band, which indicates deletion of a portion of each gene analyzed.

FIG. 3 is a western blot confirming complete loss of tapasin, tapasin-like, or B2M proteins, in distinct monoclonal cell lines (TAPBP-KO, TAPBP/TAPBPL Double-KO, B2M-KO). Lack of protein expression is the expected consequence of genetic knockout.

FIG. 4 are histograms showing retained expression of MHC class I in TAPBP-KO and TAPBP/TAPBPL Double-KO iPSCs, while B2M-KO cells completely lack MHC class I expression in the cell surface.

FIGS. 5A and 5B are boxplots showing a reduced immune response against TAPBP-KO and TAPBP/TAPBPL Double-KO iPSCs as compared to unmodified or B2M-KO iPSCs. Granzyme B (FIG. 5A) and Interferon gamma (FIG. 5B) were detected by ELISpot assays performed during admixture of TAPBP-KO, TAPBP/TAPBPL Double-KO, B2M-KO, and unmodified iPSCs with non-HLA-matched peripheral blood mononuclear cells (PBMCs) from 3 different donors.

FIGS. 6A through 6E show the insertion of a landing pad into TAPBP locus (LP-KI/TAPBP-KO). FIG. 6A is a schematic representation of the landing pad. Different combinations of primers were used to map the landing pad insertion into TAPBP locus. In FIG. 6B, primers used in genomic PCRs anneal to regions flanking the insertion site. In FIG. 6C, the forward primer anneals to the landing pad sequence, while the reverse primer anneals to part of the TAPBP locus. FIG. 6D confirms complete loss of tapasin protein via western blot and the expression of green fluorescent protein (GFP) by microscopy. FIG. 6E provides results of SSEA4 and TRA-1-60 staining (positive markers) and SSEA1 staining (negative marker) of LP-KI/TAPBP-KO monoclonal iPSCs indicative of maintained pluripotency after genetic manipulation.

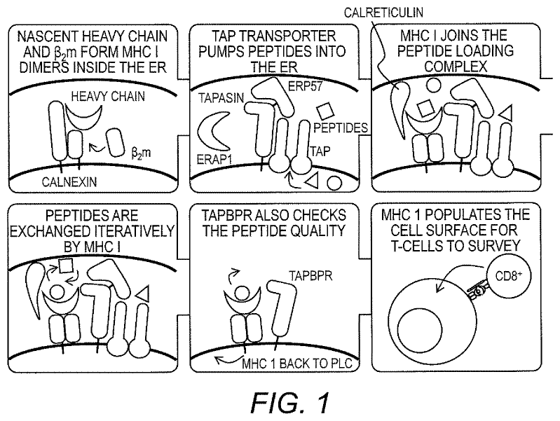

### DETAILED DESCRIPTION

The disclosure relates to methods for improving the compatibility of donor cells and tissues transplanted into unmatched recipients. Specifically, this disclosure details new methods and compositions for engineering cells such that the major histocompatibility complex type I (MHC-I) remains expressed on the cell surface and does not efficiently present antigen peptides to T lymphocytes. This approach enables engineered cells to evade recipient T cell allorecognition responses that lead to rejection of transplanted cells and tissues and simultaneously to avoid activating recipient NK cell responses strongly elicited when MHC-I is completely absent from the cell surface of donor cells.

The methods and compositions of this disclosure do not require knockout of individual HLA loci, but rather employ either knockout of a single gene or expression of one or more inhibitory proteins to impair MHC-I function without eliminating MHC-I expression. The functionally MHC-I-impaired cells cannot display antigen and lack the ability to efficiently activate unwanted recipient cytotoxic T cell allorecognition responses. Since they maintain MHC-I expression, however, they do not elicit unwanted recipient natural killer responses.

The generation of peptides and their loading on MHC class I molecules is a multistep process involving multiple molecular species that constitute the so-called antigen processing and presenting machinery (APM). The extent of T-cell activation directly correlates with the expression of several APM components, and several disease states associated with a generalized impairment of T-cell allorecognition are associated with the downregulation of specific APM components. In addition, synthetic genetic manipulations that impair MHC class 1-peptide complexes (e.g., surface expression of peptide-free MHC class I complexes) impair direct activation of T cells.

Tapasin (TAPBP) is a transmembrane glycoprotein that is a crucial component of the peptide-loading complex. It has a key role in influencing the generation of peptide repertoire and expression of stable MHC class I molecules on the cell surface. Tapasin has a chaperone-like function that stabilizes peptide transport, facilitates the removal of peptides weakly bound to MHC class I molecules and ensures high affinity peptide access to MHC class I molecules.

TAP Binding Protein-Like (TAPBPL), a homolog of tapasin, has been shown to have similar activities to inhibit MHC-I function.

For purposes of the present invention, the terms “tapasin gene” or “TAPBP gene” refer to the gene lying on human chromosome 6 that encodes Tapasin. The term “TAPBP gene region” refers to every location on human chromosome 6 that is related to expression of tapasin. The TAPBP gene region includes not only exons, but also enhancers, promoter(s), introns, basically all the regions that are correlated with either the coding sequence or regulation of tapasin expression. It is generally known that TAPBP gene comprises 8 exon regions (Herberg et al. (1998)) and the three following splice variants are reported (RefSeq, July 2008): CCDS 34426.1, CCDS 34427.2, and 34428.2. A nonlimiting example of a TAPBP gene region includes at least part of 33,299,694 bp to 33,314,387 bp region (6p21.32 band) of human chromosome 6 and flanking region thereof (see NC_000006.12 sequence).

The present invention provides methods and compositions for producing and transplanting cells which exhibit reduced antigen presentation by MHC-I by decreasing or eliminating activity of a tapasin protein or homolog thereof in the cells.

In one nonlimiting embodiment, activity of the tapasin protein or homolog thereof is decreased or eliminated by inhibiting expression of a nucleic acid sequence encoding the tapasin protein or homolog thereof. As will be understood by the skilled artisan upon reading this disclosure, various methods for inhibiting expression of a nucleic acid sequence encoding the tapasin protein or homolog thereof can be used. Nonlimiting examples include use of a gene knockout system or use of a gene editing system. Nonlimiting examples of gene editing systems which can be used include a CRISPR-Cas system, a zinc finger nuclease (ZFNs) system, or a transcription activator-like effector-based nucleases (TALEN) system.

By competing with wild-type tapasin or a homolog thereof for TAP-binding, tapasin mutants and tapasin homolog mutants function as dominant negative inhibitors. Expression of these mutants has been shown to impair MHC-I peptide loading, preventing functional display of peptides to cytotoxic T cell receptors. Accordingly, in another nonlimiting embodiment, activity of the tapasin protein or homolog thereof is decreased or eliminated by modifying the cells to express a nucleic acid sequence encoding a dominant-negative mutant of tapasin or a homolog thereof. In one nonlimiting embodiment, the cells are modified to constitutively express the A480 mutant of tapasin to block MHC-I peptide loading while maintaining MHC-I stability. In another nonlimiting embodiment, the cells are modified cells to constitutively express the N50 and N300 mutants of tapasin to block MHC-I peptide loading while partially reducing MHC-I expression.

Tapasin is also targeted by a number of viral immunoevasins, proteins that enhance the ability of viruses to remain undetected to T cell-mediated immune-surveillance via MHC-I. These proteins directly bind to tapasin and/or other components of the TAP complex to inhibit normal peptide loading of MHC-I without impairing MHC-I cell surface expression. Thus, in another nonlimiting embodiment, activity of the tapasin protein or another component of the TAP complex is decreased or eliminated by modifying the one or more cells to constitutively express a viral immunoevasin protein.

Nonlimiting examples of viral immunoevasin proteins which can be constitutively expressed include human cytomegalovirus (HCMV) US3 protein, HCMV US6 protein, adenovirus E3-19K protein, pseudorabies US3 protein, pseudorabies UL49.5 protein, herpes simplex virus ICP47 protein, equine herpesvirus (EHV) UL49.5 protein, bovine herpesvirus (BHV) UL49.5 protein, Epstein-Barr virus (EBV) BFNL2a protein, cowpox virus (CPV) CPXV12 protein, or any combinations thereof.

In one nonlimiting embodiment, the cells are modified to constitutively express the human cytomegalovirus (HCMV) US3 protein or adenovirus E3-19K protein. These proteins bind tapasin and inhibit its ability to facilitate high-affinity peptide acquisition by class I molecules. US3 also associates with both free and B2M-associated MHC-heavy chains.

In another nonlimiting embodiment, the cells are modified to constitutively express the human cytomegalovirus (HCMV) US3 protein or adenovirus E3-19K protein as well as other virus-derived immunoevasins, alone or in combination, that inhibit either tapasin or TAP function, or the function of both, including but not limited to pseudorabies US3 and UL49.5, HSV ICP47, HCMV US6, BHV and EHV UL49.5, BNFL2a (EBV), and CPXV12 (CPV).

In some nonlimiting embodiments of the present invention, the methods of the present invention may further comprise modifying the cells to introduce one or more bacteriophage integrase acceptor sites into the cellular genome.

Nonlimiting examples of cells which can be modified in accordance with the methods of the present invention include embryonic stem cells (ESCs), induced pluripotent stem cells (iPSCs), hematopoietic stem cells (HSCs), mesenchymal stem cells (MSCs), natural killer cells, natural killer cells expressing a chimeric antigen receptor (CAR-NK cells), T lymphocytes, T lymphocytes expressing a chimeric antigen receptor (CAR-T cells), or other mature cell types such as those intended for use in regenerative medicine applications, including but not limited to, skeletal muscle cells, smooth muscle cells, gastric/intestinal/colonic epithelial cells, neuroendocrine cells, endothelial cells, cardiomyocytes, hepatocytes, pancreatic islet cells, neurons, glial cells, skin cells, chondrocytes, osteoblasts, or retinal pigment epithelium cells.

The present invention also provides cells which exhibit reduced antigen presentation by MHC-I by decreasing or eliminating activity of a tapasin protein or homolog thereof in the cells.

In one nonlimiting embodiment, the cells comprise a cellular genome which lacks at least a portion of an endogenous tapasin gene or homolog thereof such that the expression of the tapasin protein or homolog thereof in the cell is decreased or eliminated.

In another nonlimiting embodiment, the cells comprise a cellular genome modified to express a nucleic acid sequence encoding a dominant-negative mutant of tapasin or a homolog thereof and/or a nucleic acid sequence encoding viral immunoevasin protein. Nonlimiting examples of immunoevasin proteins which can be encoded by the cells include human cytomegalovirus (HCMV) US3 protein, HCMV US6 protein, adenovirus E3-19K protein, pseudorabies US3 protein, pseudorabies UL49.5 protein, herpes simplex virus ICP47 protein, equine herpesvirus (EHV) UL49.5 protein, bovine herpesvirus (BHV) UL49.5 protein, Epstein-Barr virus (EBV) BNFL2a protein, cowpox virus (CPV) CPXV12 protein or any combinations thereof.

Nonlimiting examples of cells which can be modified in accordance with the present invention include embryonic stem cells (ESCs), induced pluripotent stem cells (iPSCs), hematopoietic stem cells (HSCs), mesenchymal stem cells (MSCs), natural killer cells, natural killer cells expressing a chimeric antigen receptor (CAR-NK cells), T lymphocytes, T lymphocytes expressing a chimeric antigen receptor (CAR-T cells), or other mature cell types such as those intended for use in regenerative medicine applications, including but not limited to, skeletal muscle cells, smooth muscle cells, gastric/intestinal/colonic epithelial cells, neuroendocrine cells, endothelial cells, cardiomyocytes, hepatocytes, pancreatic islet cells, neurons, glial cells, skin cells, chondrocytes, osteoblasts, or retinal pigment epithelium cells.

The present invention also provides nucleic acid sequences and vectors comprising these nucleic acid sequences for use in inhibiting expression or function of a tapasin protein or homolog thereof in a host cell.

In one nonlimiting embodiment, the nucleic acid sequence comprises a 5′ homology arm complementary a tapasin gene or a portion thereof or homolog thereof, a 3′ homology arm complementary to a tapasin gene or portion thereof or homolog thereof, and at least one exogenous polynucleotide. In one nonlimiting embodiment, the 5′ homology arm and the 3′ homology arm are complementary to different parts of a tapasin gene region of a host cell.

The “homology arms” flank the exogenous polynucleotide of interest and contain a sequence that is homolog to flanking region of a chromosome-insertion site. The 5′ and 3′ homology arms determine the location of insertion for the nucleic acid fragment of the present invention. The homology arm can comprise a base sequence 50 to 1,000 bp, more specifically 100 to 1,000 bp, or 500 or 800 bp in length. Sequences substantially identical (e.g. more than 60%, 80%, 90%, 95% or 99% identity) to the flanking region of the chromosome-insertion site can be used for the homology arms of the present invention. Those skilled in the art can easily design and synthesize suitable homology arm sequences for various applications.

In one nonlimiting embodiment, the 5′ and 3′ homology arms are nucleic acid fragments with complementary sequences to an adjacent chromosomal transfer site that is present within in human chromosome 6 (NCBI Reference Sequence (NC_000006.12). In one nonlimiting embodiment, the nucleic acid sequence is designed for insertion in any bp position 33,297,694 to 33,319,212 of human chromosome 6 (NC_000006.12). More specifically, in this nonlimiting embodiment, the 5′ and 3′ homology arms can be homologous to any bp position 33,297,694 to 33,319,212 of human chromosome 6 (NC_000006.12).

In one nonlimiting embodiment, the nucleic acid sequence the 5′ and/or 3′ homology arms exhibits at least 60%, more preferably at least 70%, more preferably at least 80%, more preferably at least 90% or higher sequence identity with a nucleic acid sequence encoding tapasin or portion thereof as set forth in SEQ ID Nos 14-23 or homolog thereof such as set forth in SEQ ID NO:24 or 25.

In one nonlimiting embodiment, the nucleic acid sequence further comprises a portion of an exon region of the tapasin genome or a homolog thereof.

By “complementary” as used herein it is meant a nucleic acid sequence with at least 60%, more preferably at least 70%, more preferably at least 80%, more preferably at least 90% or higher sequence identity with a tapasin gene or portion thereof or homolog thereof.

The term “exogenous polynucleotide”, herein, refers to polynucleotide a host cell does not carry endogenously. Nucleic acid sequences of the present invention can be inserted into a chromosome of a host cell via homology arms. Exogenous polynucleotides can work as a chromosomal landing pad or be a polynucleotide of interest to be expressed.

The term “polynucleotide of interest” (or “gene of interest” or “target gene”) is intended to include a cistron, an open reading frame (ORF), or a polynucleotide sequence which codes for a polypeptide or protein product (“polypeptide of interest” or “target polypeptide”). The polynucleotide of interest can be included in the nucleic acid sequence containing the homology arms. For stable integration and expression in an engineered host cell bearing a chromosomal landing pad described herein, a polynucleotide of interest can additionally contain appropriate transcription regulatory elements (e.g., promoter sequences) operably linked to the coding sequence and also a cognate site-specific recombination sequence (e.g., attB or attP site). Various target polypeptides can be encoded by and expressed from a polynucleotide of interest, e.g., therapeutic proteins, nutritional proteins and industrial useful proteins.

In one nonlimiting embodiment, the exogenous polynucleotide comprises a site-specific recombinant sequence recognized by a site-specific recombinase. As a nonlimiting example, the site-specific recombination sequence can be a recognition sequence recognized by a phage integrase, such as the attP site or the attB site recognized by phiC-31 phage integrase. Additionally, the exogenous polynucleotide may include a sequence that directly encodes a protein.

In some nonlimiting embodiments, the exogenous polynucleotide to be integrated into the host genome comprises a site-specific recombination sequence that is recognized by a unidirectional site-specific recombinase and supports the site-specific recombination of, for example, a phage integrase such as phiC-31 phage integrase. In one nonlimiting embodiment, the site-specific recombination sequence comprises a phage attachment site (e.g., attP site) or a bacterial attachment site (e.g., attB site) recognized by an integrase (e.g., a tyrosine integrase or a serine integrase). Examples of such sequences include, but are not limited to, attB and attP sequences (as well as pseudo att sites) recognized by several phage integrases, e.g., phiC-31 integrase or X integrase. Suitable recombination sites also include sequences that are recognized by mutant integrases. During the integration of the phage genome into the genome of its host (e.g., an E. coli cell), the enzyme catalyzes the DNA exchange between the attP site of the phage genome and the attB site of the bacterial genome, resulting in the formation of attL and attR sites. By inserting into the host genome (e.g., at the native chromosomal integration sites disclosed herein) a site-specific recombination site (e.g., attP site) that is recognized by a phage integrase (e.g., phiC-31 integrase), an exogenous polynucleotide attached to the cognate recognition site (e.g., attB site) can be readily inserted into the host genome via site-specific recombination catalyzed by the phage integrase. The phage attachment site (attP) and the bacterial attachment site (attB site) recognized by any site-specific recombinase (e.g., serine or tyrosine phage integrases) may be employed as the site-specific recombination sequence described herein. These include both the wildtype (native) attB and attP sites recognized by a given phage integrase as well as pseudo sites. Site-specific recombinases and their respective recognition sequences (attP and attB sites) for various phages and other species have been known and characterized in the art. Nonlimiting examples include phage integrase (Enquist et al., Cold Spring Harbor Symp. Quant. Biol. 43:1115-1120, 1979), Bxb1 phage integrase (REF: doi 10.2144/000112150), HK022 phage integrase (Yagil et al., J. Mol. Biol. 207:695-717, 1989), P22 phage integrase (Leong et al., J. Biol. Chem. 260:4468-4477, 1985), HP1 phage integrase (Waldman et al., J. Bacteriol. 165:297-300, 1986), L5 phage integrase (Lee et al., J. Bacteriol. 175:6836-6841, 1993), phiC-31 phage integrase (Kuhstoss and Rao, J. Mol. Biol. 222:897-908, 1991), R4 phage (Groth et al., Proc. Natl. Acad. Sci. USA 97:5995-6000, 2000), TP901 phage integrase (Christiansen et al., J. Bacterial. 178:5164-5173, 1996), γδ transposon resolvase (Reed et al., Nature 300:381-383, 1982), Tn3 transposon resolvase (Krasnow et al., Cell 32:1313-1324, 1983) and Mu phage invertase Gin (Kahmann et al., Cell 41:771-780, 1985).

Other than wild type recombination sites that are recognized by site-specific recombinases, the site-specific recombination sequence present in the nucleic acid sequences or vectors for landing pad insertion can also comprise a sequence that is different from the wild-type recognition site (e.g., wild type attP site) by at least one base pair alteration (a substitution, deletion or insertion). Sequence alterations may be at any position within the site-specific recombination sequence. In some embodiments, the modified site-specific recombination sequences have multiple sequence alterations as compared to a wild type recognition site. When such a modified site-specific recombination sequence (e.g., a modified attP site) is integrated into the genome of an engineered host cell as described herein, the wild type or mutant version of the corresponding integrase (e.g., a mutant phi-C31 integrase) may be needed in order to incorporate an exogenous polynucleotide or transgene into the recombination site. Various mutant integrases (e.g., mutant phiC-31 integrase) are also known in the art. See, e.g., Smith et al., Nuc. Acids Res. 32, 2607-2617, 2004; and Kevarala et al., Mol. Ther. 17, 112-120, 2008.

In one nonlimiting embodiment, the exogenous polynucleotide encodes a chimeric antigen receptor (CAR). A host cell transformed with such exogenous polynucleotide can be utilized as a CAR-X therapeutic, wherein the X can be any immune cell type, including NK cell or T cell.

The term “chimeric antigen receptor” or “CAR” refers to a hybrid protein or polypeptide synthetically engineered to include antigen binding domain (for example, single-chain variable fragment (scFv)) linked to an activation domain for immune cells (such as NK cells or T-cells). Immune cells equipped with a chimeric antigen receptor can have specificity and reactivity redirected to the selected target in a non-MHC restricted fashion. Non-MHC-restricted antigen recognition provides the immune cell expressing the CAR a capability to recognize the antigen regardless of the antigen processing, therefore evading the main mechanism of immune escape of tumor cells. Furthermore, CAR advantageously does not multimerize with innate T-cell receptor α and/or β chain(s).

A chimeric antigen receptor of the present invention can comprise a binding domain for an antigen that are expressed on cancer cells hence targeted by CAR-T therapeutics in the art. Non-limiting examples for the antigen-binding domain comprise any antibody or the binding fragment thereof chosen from anti-EGFR antibodies, anti-Her2 antibodies, anti-EGFRvIII antibodies, anti-CD33 antibodies, anti-CLL-1 antibodies, anti-CEA antibodies, anti-CD19 antibodies, anti-CD22 antibodies, anti-BCMA antibodies, anti-Claudin-3 antibodies and anti-CS1 antibodies.

In one nonlimiting embodiment, the chimeric antigen receptor of the present invention binds to claudin-3. The term “claudin-3”, also written as CLDN3, is a protein member of claudin family that plays specific roles of removing the space between two adjacent cells at a tight junction. Tight junctions are rigid structure connecting neighboring cellular membranes in organs in organisms such as animals. Claudin-3 is a structural protein that regulates the permeability of solutes such as ions across the sheet of cells connected with the protein. Claudin-3 comprises 4 transmembrane domains with two peptide loops exposed to the extracellular space. Between the two extracellular loops, the one closer to the N-terminus of claudin-3 protein is called the first extracellular loop (referred to as ECL-1 or EL1 in the present invention), while the other is named the second extracellular loop (ECL-2 or EL2). Claudin-3 can be specifically exposed in solid cancer cells.

In one nonlimiting embodiment, a CAR of the present invention may comprise a transmembrane domain. The transmembrane domain can be derived from either a natural or synthetic donor known in the art. The transmembrane domain can be a transmembrane domain from a protein chosen from a non-limiting group comprising T-cell receptor, CD27, CD28, CD3, CD45, CD4, CD5, CD8(CD8a), CD9, CD16, CD22, CD33, CD37, CD64, CD80, CD86, CD134, CD137 and a, p or chain of CD154. In one nonlimiting embodiment, a CAR of present invention comprises a transmembrane domain comprising a hinge from CD8.

In one nonlimiting embodiment, a CAR of the present invention comprises a signaling domain. The term “intracellular signaling domain” refers to a functional domain of a protein that operates by transducing information inside a cell to induce cellular activity via certain signaling pathway by working as an effector that generates second messenger or react to a secondary messenger. The “cellular activation” refers to an increase of an activity of a cognate cell, wherein the activation can be, for example, but not limited to, stimulation of immune response of a cell, which is not specifically limited. When the cell is an immune cell, the activation can comprise stimulation of an immune response of the cell per se as well as an increase in number of the immune cells. The intracellular signaling domain is not limited as long as the domain can induce the activation of the cell, in particular an immune cell, upon binding of antigen binding to the extracellular antigen binding domain. Many different intracellular signaling domains can be used. Nonlimiting examples include Immunoreceptor tyrosine-based activation motif (ITAM), such as ITAM derived from one or more of CD3ξ, TCRξ, FcRγ, FcRβ, CD3γ, CD3δ, CD3ε, CDS, CD22, CD79a, CD79b, CD278, CD66d, DAP10, DAP12, FcRε (in particular γ) and any combination thereof. In a nonlimiting embodiment, a CAR of the present invention comprises an intracellular signaling domain derived from CD3ζ.

CAR in the present invention may further comprise an additional intracellular signaling domain(s) and a costimulatory domain(s) depending on the cell type the CAR is expressed in.

A costimulatory domain herein, which is derived from an intracellular signaling domain derived from a costimulatory molecule, transduces maximal activation signal of the host cell, in particular immune cells, adding to the primary signal generated by the signaling domain, as an intracellular part of a CAR of the present invention. For example, some immune cells, such as T lymphocytes and NK cells, require 2 types of signal including a primary activation signal and a costimulatory signal for maximal activation. Hence, a CAR can comprise a costimulatory domain to induce both of the signals upon binding of the extracellular domain of the CAR.

The costimulatory molecule refers to a cell surface molecule that is required for sufficient response to an antigen by immune cells. The costimulatory molecule can be chosen from a group of nonlimiting examples comprising MHC class I molecules, TNF receptor proteins, Immunoglobulin-like proteins, cytokine receptors, integrins, SLAM proteins (signaling lymphocytic activation molecules), NK cell activating receptors, BTLA, Toll ligand receptor, OX40, CD2, CD7, CD27, CD28, CD30, CD40, CDS, ICAM-1, LFA-1(CD11a/CD18, lymphocyte function-associated antigen-1), 4-1BB(CD137), B7-H3, CDS, ICAM-1, ICOS(CD278), GITR, BAFFR, LIGHT, HVEM(LIGHTR), KIRDS2, SLAMF7, NKp80(KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1(CD226), SLAMF4(CD244, 2B4), CD84, CD96(Tactile), CEACAM1, CRTAM, Ly9(CD229), CD160(BY55), PSGL1, CD100(SEMA4D), CD69, SLAMF6(NTB-A, Ly108), SLAM(SLAMF1, CD150, IPO-3), BLAME(SLAMF8), SELPLG(CD162), LTBR, LAT, GADS, SLP-76, PAG/Cbp, CD19a, CD83-specific ligand, PD-1 and any combination thereof. The costimulatory domain can be an intracellular part of the chosen costimulatory molecule. In one nonlimiting embodiment of the present invention, a costimulatory domain of a CAR can comprise SLAMF4 (CD24, 2B4).

The costimulatory domain can be linked to either the N-terminal or C-terminal direction to the signaling domain(s), or in between multiple signaling domains.

In one embodiment of the present invention, the CAR further comprises a CD8 leader sequence at the N-terminus.

The nucleic acid sequences of the present invention serve as a landing pad, which lies in TAPBP gene region of a host cell and provides an insertion site for a nucleic acid construct for safe transformation of the host cell. Furthermore, the insertion leads to knockout of tapasin in the host cell preventing the “missing-self” response mediated by NK cells as well as to hinder antigen peptide presentation avoiding T cell-mediated cytotoxicity, resulting in excellent immunocompatibility of the host cell.

In some embodiments, the nucleic acid sequence comprising the exogenous polynucleotide(s) can be inserted into the TAPBP gene region of a host cell, wherein the insertion can lead to separation or deletion of at least a part of the original TAPBP gene region. The deletion can be derived naturally by the insertion of the nucleic acid sequence or via an additional deletion process. Without being bound to any particular theory, it is believed that when the nucleic acid sequence of the present invention is transformed into a host cell, at least part of TAPBP gene region of the host cell is deleted so that the host cell presents reduced antigen presentation by MHC class I. Furthermore, the newly inserted exogenous polynucleotide of interest can exhibit an intended function(s), including serving as a landing pad or expressing a protein of interest. This function(s) can occur without hindering the original activities of the host cell, making the nucleic acid sequence of the present invention a vector for safe delivery of genetic materials. In other words, after being inserted into the host cell, the nucleic acid sequence can inhibit the expression and/or function of tapasin in the host cell while inducing expression of the inserted gene(s) of the exogenous polynucleotide of interest.

Vectors for use in the present invention can be selected from any commercially available expression vectors. Vectors of the present invention can be transferred to a safe harbor in a host genome. Vectors can be DNA, RNA or plasmid, and can be viral vectors such as lentiviral vector, adenoviral vector or retroviral vector. Vectors for use in the present invention may comprise a promotor(s) to regulate expression of a protein(s). Vector for use in the present invention can, other than a chimeric antigen receptor(s), further comprise constructs for expression of factor(s) for differentiation or growth of the transformed cells.

In one nonlimiting embodiment, the vector used in the present invention is a donor vector, which can contain attB, attP, attL or attR. After integrating the donor vector into the host genome, the gene of interest can be inserted into the location of the donor vector integration using site-specific recombinases, such as bacteriophage integrase.

The nucleic acid sequences and vectors of the present invention can be readily constructed in accordance with standard procedures known in the art of molecular biology (e.g., Sambrook et al. and Brent et al.) and the disclosure herein. To generate the vectors, the above-described nucleic acid sequences comprising the homology arms and the exogenous polynucleotide sequence (e.g., a transgene or a chromosomal landing pad) can be inserted into various known plasmids for transfecting mammalian host cells. Such known plasmids include, e.g., BPV, EBV, vaccinia virus based vector, SV40, 2-micron circle, pcDNA3.1, pcDNA3.1/GS, pYES2/GS, pMT, p IND, pIND(Sp1), pVgRXR (Invitrogen), and the like, or their derivatives. These plasmids are all described and well known in the art (Botstein et al., Miami Wntr. SyTnp. 19:265-274, 1982; Broach, In: The Molecular Biology of the Yeast Saccharomyces: Life Cycle and Inheritance, Cold Spring Harbor Laboratory, Cold Spring Harbor, N.Y., p. 445-470, 1981; Broach, Cell 28:203-204, 1982; Dilon et al., J. Clin. Hematol. Oncol. 10:39-48, 1980; and Maniatis, In: Cell Biology: A Comprehensive Treatise, Vol. 3, Gene Sequence Expression, Academic Press, NY, pp. 563-608, 1980).

The nucleic acid sequences and vectors comprising the nucleic acids can be incorporated into mammalian host cells to inhibit expression or function of tapasin or a homolog thereof in the host cell. Further, these nucleic acid sequences and vectors can be used in in-vitro and ex-vivo methods for integrating and expressing an exogenous polynucleotide in a mammalian host cell, and enhancing the immunocompatibility of the mammalian host cell, comprising inserting at least one exogenous polynucleotide of interest into a TAPBP gene region of the isolated host cell.

In accordance with the methods of the present invention, TAPBP-KO iPSCs were generated via a CRISPR/Cas9 methodology using guide RNAs for CRISPR gene editing (pLentiCRISPR vector—Puromycin resistance): TAPBP exon 2/3.

In accordance with methods of the present invention, TAPBP/TAPBPL Double-KO iPSCs were also generated via CRISPR/Cas9 methodology using guide RNAs for CRISPR gene editing (pLentiCRISPR vector—Puromycin resistance): TAPBP exon 2/3 and TAPBPL exon 3/4.

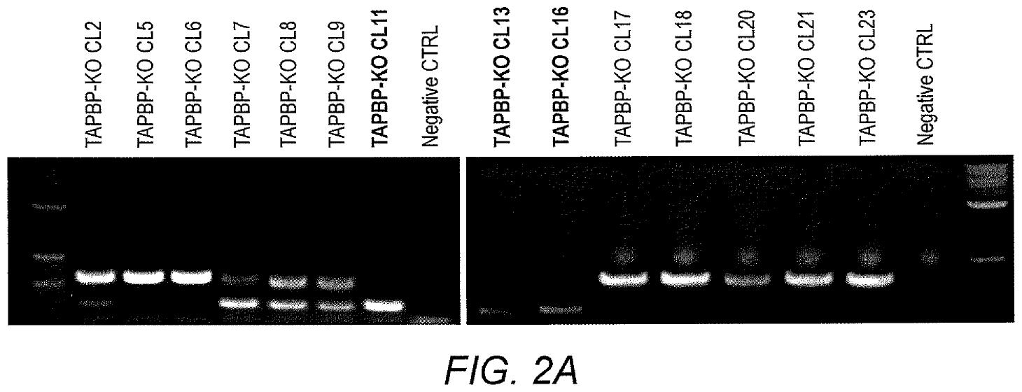

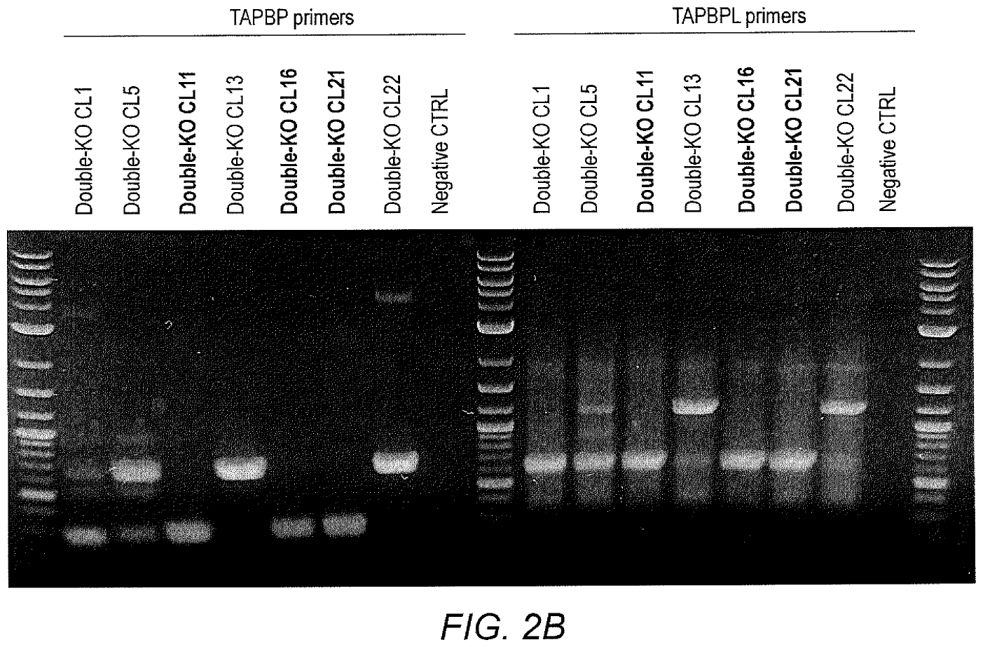

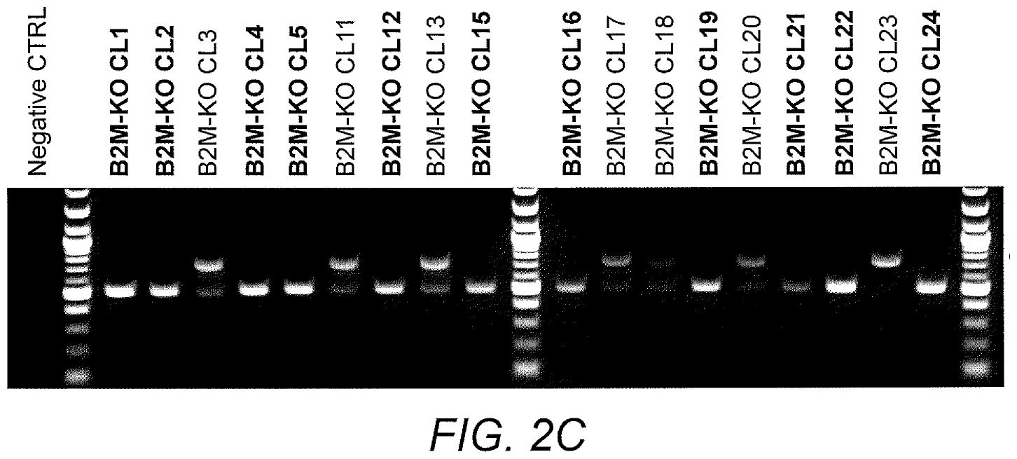

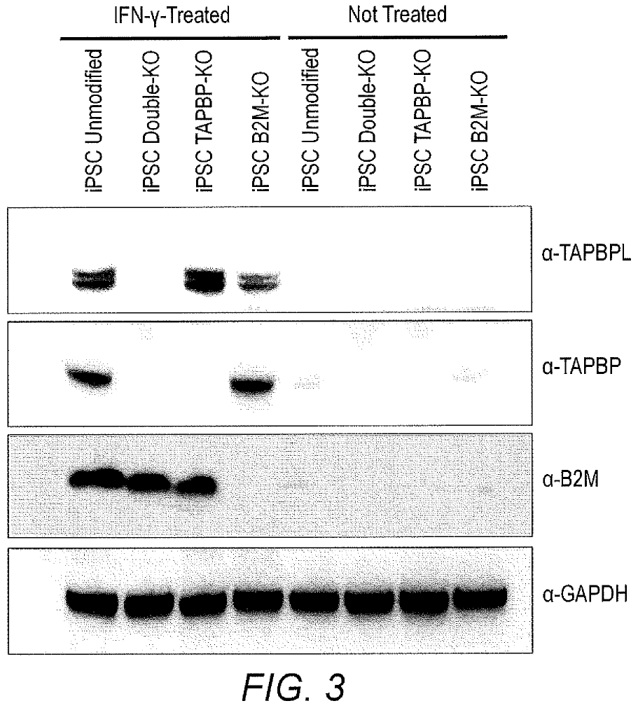

After electroporation, antibiotic selection and evaluation of gene editing efficiency, polyclonal cells were subjected to limiting dilution. Monoclonal cells were obtained by limiting dilution. The TAPBP-KO and TAPBP/TAPBPL Double-KO monoclonal iPSCs were further analyzed by genomic PCR screening (FIGS. 2A and 2B) and confirmation of abolishment of TAPBP and TAPBPL expression in clones was obtained by western blotting (see FIG. 3).

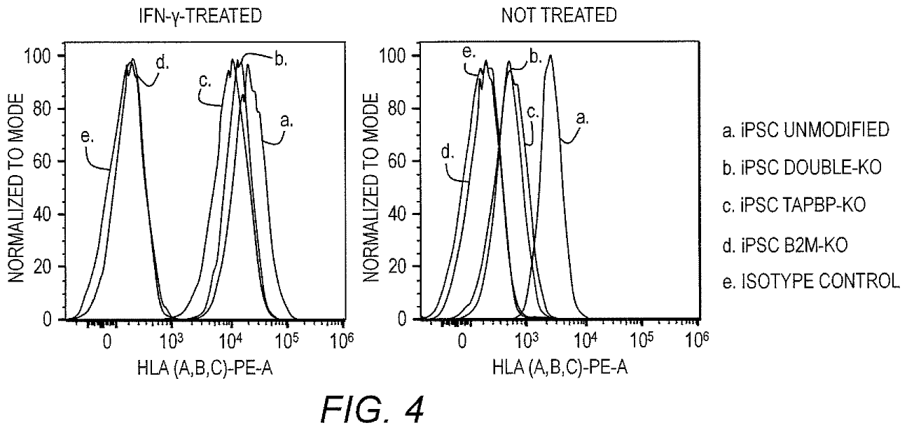

Further, functional analyses confirmed that the TAPBP-KO and TAPBP/TAPBPL Double-KO iPSCs were shown to express MHC-I after interferon gamma treatment (FIG. 4). Additionally, the clones of TAPBP-KO and TAPBP/TAPBPL Double-KO iPSCs keep pluripotency after genetic manipulation. The TAPBP-KO iPSCs also successfully differentiated to cardiomyocytes.

Thus, the TABBP-KO and TAPBP/TAPBPL Double-KO iPSCs produced in accordance with the present invention have been confirmed to express MHC-I, keep pluripotency after genomic manipulation, and efficiently differentiate to other cell types.

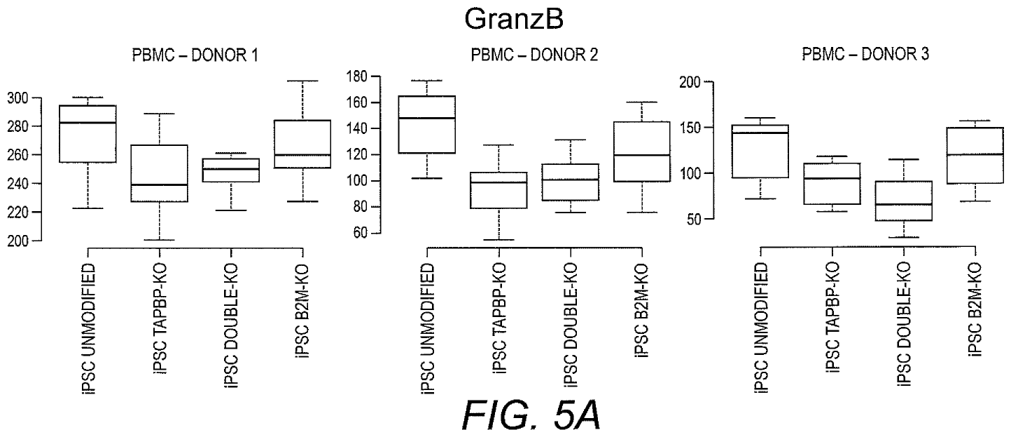

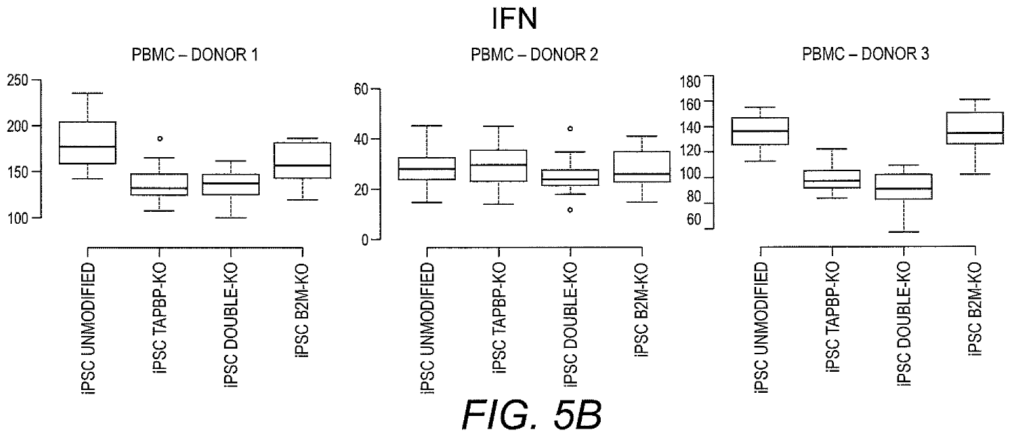

Further, immune cells showed reduced activation against TAPBP-KO and TAPBP/TAPBPL Double-KO iPSCs when compared to B2M-KO and TAPBP-expressing iPSCs with two markers for immune cell activation, specifically granzyme B (GranB), a classic NK cell activation marker also secreted by activated cytotoxic T cells and macrophages (see FIG. 5A) and interferon gamma (IFNγ), a classic cytotoxic T cell activation marker (see FIG. 5B).

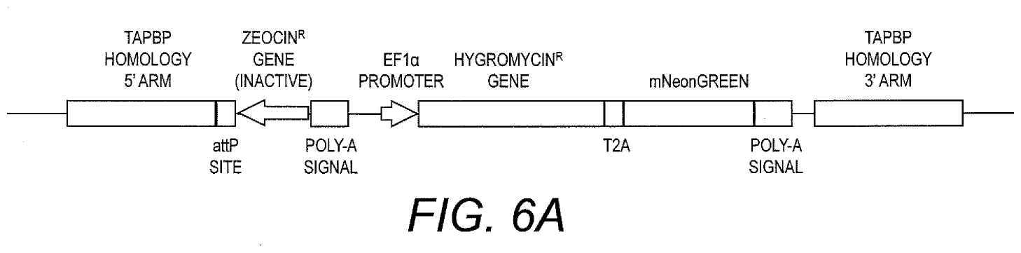

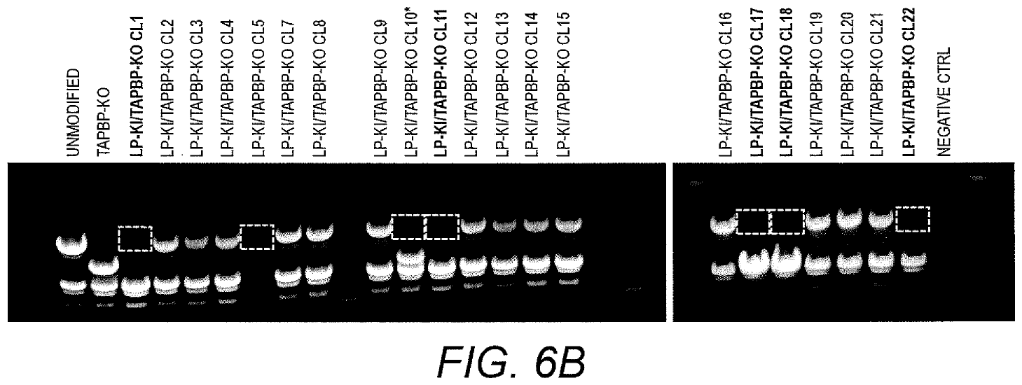

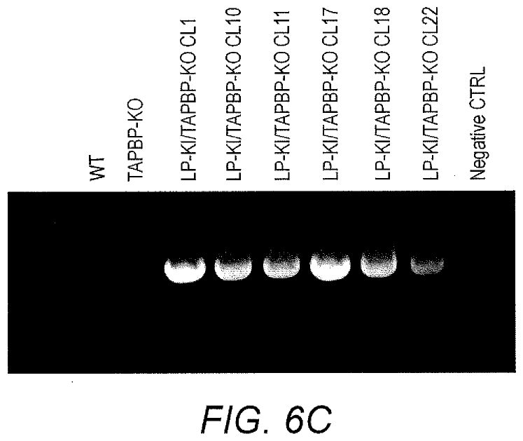

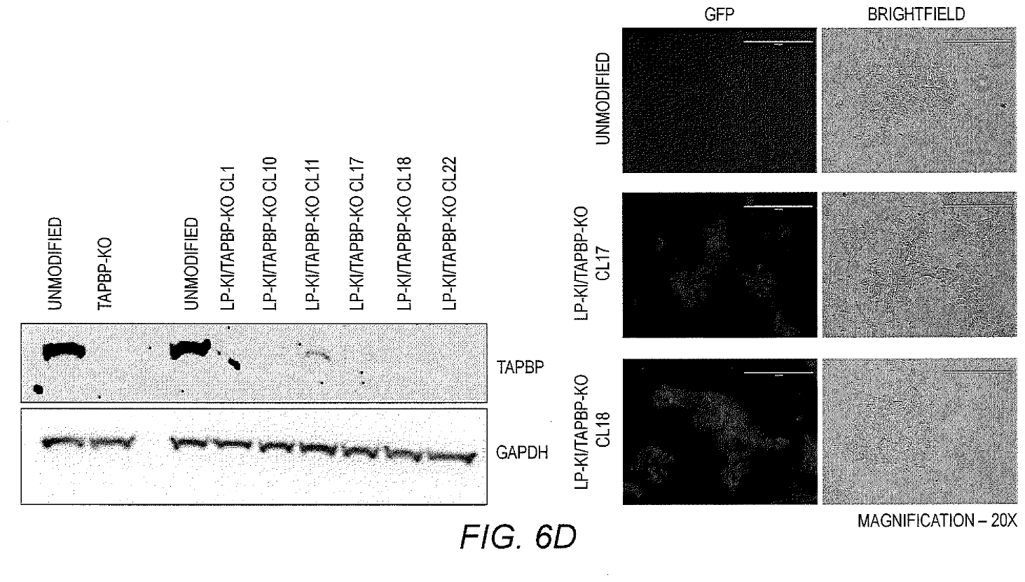

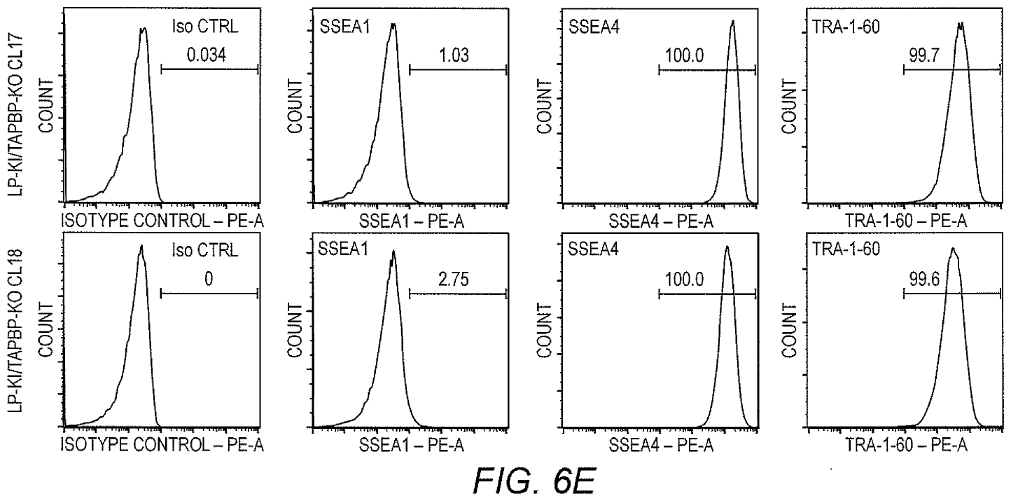

Five additional iPSC clones that are LP-KI/TAPBP-KO have been produced in accordance with the compositions and methods disclosed herein. In these cells, a specific DNA sequence (landing pad) was inserted into TAPBP locus. A schematic representation of the inserted landing pad is shown in FIG. 6A. Different combinations of primers were used to map the landing pad insertion into TAPBP locus. In FIG. 6B, primers used in genomic PCRs anneal to regions flanking the insertion site. In FIG. 60, the forward primer anneals to the landing pad sequence, while the reverse primer anneals to part of the TAPBP locus. LP-KI/TAPBP-KO clones lack tapasin expression, while expressing green fluorescent protein (GFP), a reporter gene present in the inserted landing pad (see FIG. 6D). Additionally, LP-KI/TAPBP-KO iPSC clones keep pluripotency after genetic manipulation (FIG. 6E) as assessed by SSEA4 and TRA-1-60 staining (positive markers) and SSEA1 staining (negative marker).

Thus, as demonstrated herein by these experiments, the methodologies of this invention make it possible to take cells from a single donor and modify them so that they are non-immunogenic and can be transplanted into multiple unmatched recipients with fewer unwanted clinical side effects and longer survivability of the transplant in the recipient. These immune-compatible cells produced in accordance with the present invention will be useful in any regenerative medicine application that benefits from use of immune-compatible cells for transfer into unmatched recipients and can replace engineered immune cell therapies such as CAR-NK and CAR-T, which at present time rely on use of autologous cell transplants engineered on an individualized patient-by-patient basis. These methods are highly expensive and potentially unfeasible for use in routine clinical settings. In contrast, the methods of the present invention can be applied to engineering of immune-compatible pluripotent stem cells or embryonic stem cells that will enable in vitro functionalization of engineered cells such that immune-compatibility is improved while simultaneously enabling cells by insertion of bacteriophage integrase receiver sites that enable rapid, reliable, site-directed knock-in without off-target genome damage. In summary, the methods, cells and compositions of the present invention provide a means for development of so-called ‘off-the-shelf’ transplants suitable for industrial-scale production and distribution at far lower costs and with less technical complexity than current patient-by-patient individualized autologous products.

The following nonlimiting examples are provided to further illustrate the present invention.

### EXAMPLES

### Example 1

Genetic Knockout of Tapbp (Tapasin) and Tapbpl (Tapasin-Like) in Effector Cells or Effector Cell Progenitor (Including iPSCs, ESCs, Hematopoietic Stem Cells)
Cells: iPSCs were obtained from RUCDR Infinite Biologics, Cell line ID: NN0005200 (CD34+from cord blood, episomally reprogramed). Cells were cultured in mTeSR Plus media (StemCell Technologies), with fresh media change every other day. Once 80%-90% confluent, cells were dissociated using Gentle Cell Dissociation Reagent (StemCell Technologies) or ReLeSR™ (StemCell Technologies) and seeded in plates coated with Vitronectin (VTN-N) Recombinant Human Protein (Gibco). For cardiomyocyte differentiation and maintenance, iPSCs were cultured in media from the STEMdiff™ Cardiomyocyte Differentiation Kit and STEMdiff™ Cardiomyocyte Maintenance Kit (StemCell Technologies), following manufacturer's protocol.
Generation of TAPBP-KO cells: Five micrograms of DNA—pLentiCRISPR TAPBP RNA guide 2 and pLentiCRISPR TAPBP RNA guide 4 (GenScript, cat numbers SC1805 and SC1678) were used to electroporate iPSCs using the Neon™ Transfection System (Life Technologies), following manufacturer's instructions. Electroporation conditions: 1200V/20 ms/2 pulses. Puromycin selection (0.1 μg/mL) started 24 hours after electroporation and lasted about 5 days.
Generation of TAPBP/TAPBPL Double-KO cells: Five micrograms of DNA—pLentiCRISPRv2 plasmid with the following TAPBP gRNA target sequences: GAACCAACACTCGATCACCG (SEQ ID NO:1), GCCCCGGGGATACCGCCTGA (SEQ ID NO:2); pLentiCRISPRv2 plasmid with the following TAPBPL gRNA target sequences: GGTGCGCACCGTCCTTCGCC (SEQ ID NO:3) and CTCAGTGGCAAGTTCAGCGT (SEQ ID NO:4), and pLentiCRISPRv2 plasmid with the following B2M gRNA target sequences: TCACGTCATCCAGCAGAGAA (SEQ ID NO:5) and CACAGCCCAAGATAGTTAAG (SEQ ID NO:6)—were used to electroporate iPSCs using the Neon™ Transfection System (Life Technologies), following manufacturer's instructions. Electroporation conditions: 1200V/20 ms/2 pulses. Puromycin selection (0.1 μg/mL) started 24 hours after electroporation and lasted about 5 days.
Serial limiting dilution: Single cell suspensions from polyclonal puromycin-resistant iPSCs were obtained by harvesting cells using Gentle Cell Dissociation Reagent (StemCell Technologies). To obtain monoclonal TAPBP-KO, TAPBP-TAPBPL Double-KO or B2M-KO iPSCs, dilution cloning was performed in vitronectin-coated 96-well plates. Cloning medium was as follows: 1× mTeSR Plus, 1× RevitaCell™ Supplement (Gibco), 1× CloneR™ (StemCell Technologies). 100 μl cloning medium was added to all wells except well A1. Then, 200 μl of cell suspension at 2×10⁴ cells/mL was added to well A1. To perform a 1/2 dilution series, re-suspensions of 100 μL were performed down the column (wells B1-H1). 100 μl from well H1 was discarded. Next, 100 μl of fresh cloning medium was added to all wells in column 1 (wells A1-H1). A second-round of 1/2 dilution series was performed by re-suspending 100 μl down the rows (1-12). 100 μL from row 12 was discarded. Finally, 100 μL of cloning medium was added to all wells and plates were kept at 37° C. and 5% CO₂ until single colonies were grown enough to be expanded to a 24-well plate. Medium was replaced every 2 days (mTeSR Plus+1× RevitaCell™).
Genomic PCR: Genomic DNA from puromycin-resistant iPSCs, TAPBP-KO, TAPBP/TAPBPL Double-KO or B2M-KO clones were extracted using GeneJET Genomic DNA Purification Kit (ThermoFisher). PCR reactions were performed using Phusion® High-Fidelity PCR Master Mix with GC Buffer (NEB) and specific primers as follows: to detect TAPBP deletion (Forward 5′-AGCGCCATGAAGTCCCTGTCTCTGCTC-3′ (SEQ ID NO:7) and Reverse 5′-GGCACGAAGCGGCTCATCTCGCAG-3′(SEQ ID NO:8)), TAPBPL deletion (Forward 5′-GCAAATCCAGGACTTTAACTCTCACC-3′ (SEQ ID NO:9) and Reverse 5′-GAGATGGAGTTTCGCTCTTGTTGCC-3′(SEQ ID NO:10)), and B2M deletion (Forward 5′-GGGAAGGTGGAAGCTCATTT-3′ (SEQ ID NO:11) and Reverse 5′-CTAGAGGAAGCCAGTAGGTAAGA-3′ (SEQ ID NO:12).
Western blot: Protein extracts of TAPBP-KO, TAPBP/TAPBPL Double-KO, B2M-KO and unmodified iPSCs were prepared 48 hours after treatment with 50 ng/mL Interferon gamma. About 30 μg protein extract of each sample was submitted to SDS-PAGE and transferred to PVDF membranes. After blocking, membranes were incubated overnight with rabbit anti-TAPBP (Bolter Biological), mouse anti-B2M (BioLegend), mouse anti-TAPBPL (ThermoFisher) and mouse anti-GAPDH (Proteintech) antibodies. Membranes were washed and incubated with goat anti-rabbit IgG (Buster Biological) and goat anti-mouse IgG (ThermoFisher) antibodies, both HRP-conjugated. Signal was detected by using ECL Western Blotting Substrate (Pierce)
Flow cytometry: to check MHC class I expression, TAPBP-KO, TAPBP/TAPBPL Double-KO, B2M-KO and unmodified iPSCs were treated with 50 ng/mL Interferon gamma for 48 hours. Cells were then harvested using Gentle Cell Dissociation Reagent (StemCell Technologies) and stained with PE-conjugated anti-HLA-ABC antibody (clone W6/32, Invitrogen) or PE-conjugated IgG2a kappa Isotype Control (clone eBM2a, Invitrogen) antibodies. To check the expression of pluripotency markers, LP-KI/TAPBP-KO clones were harvested using Gentle Cell Dissociation Reagent (StemCell Technologies) and stained with PE-conjugated anti-TRA-1-60-R (clone TRA-1-60-R, Biolegend), PE-conjugated anti-SSEA-1 (clone MC-480, Biolegend), FITC-conjugated anti-SSEA-4 (clone MC-813-70, Biolegend), PE-conjugated anti-Mouse IgM Isotype control (clone MM-30, Biolegend), and FITC-conjugated anti-Mouse IgG3 Isotype control (clone MG3-35, Biolegend) antibodies. Samples were submitted to flow cytometry using an Attune™ NxT flow cytometer (ThermoFisher) and collected data was analyzed using FlowJo software.
ELISpot assay: iPSCs were seeded at 95% confluency in 96-well plates prior to incubation with human peripheral blood mononuclear cells (hPBMCs). Prior to ELISpot, TAPBP-KO, TAPBP/TAPBPL Double-KO, B2M-KO and unmodified iPSCs were treated with 50 ng/mL interferon gamma for 48 hours to stimulate MHC class I expression. After conditioned media removal, followed by wash with DPBS, target iPSCs were incubated with thawed hPBMCs and 10 U/mL IL-2 for 24 hours. Cryopreserved hPBMCs were obtained from Cellular Technology Limited (CTL) and thawed following manufacturer's protocol. The next day, activated hPBMCs were transferred to pre-coated ELISpot plates (Human IFN-γ and GranzB Double-color enzymatic assay, ImmunoSpot), where they were allowed to secret IFN-γ and GranzB for 24 hours at 37° C. and 5% CO₂. ELISpot plate were developed 24 hours later following manufacturer's protocol. Plates were sent back to CTL to be scanned and have number of IFN-γ and GranzB spots per well counted.

### Example 2

### Insertion of One or More Bacteriophage Integrase

It is advantageous to functionalize effector cells and other engineered cells by insertion of one or more bacteriophage integrase ‘acceptor sites’ (including but not limited to R4 integrase attP and PhiC31 integrase attP), that enables high-efficiency insertion of DNA expression/selection cassettes that produce gene products (mRNA and proteins) of interest. Accordingly, for this embodiment, a TAPBP-KO approach such as described in Example 1 is combined with knock-in of such a cassette so as to simultaneously knock-out tapasin expression and knock-in the receiver.

### Example 3

### Specific Cytotoxic T and NK Cells Immune-Response to Tapasin Null-Engineered Cells

Cytotoxic T cells: The ability of the TAPBP-KO or TAPBP/TAPBPL Double-KO iPSCs to evade recognition by non-HLA-matched cytotoxic T cells is tested by measuring in vitro cell activation following co-culture with iPSCs. TAPBP-KO, TAPBP/TAPBPL Double-KO, 132M-KO, and unmodified iPSCs are seeded in 96-well plates and then treated for 2 days with IFN-γ to stimulate MHC-I expression. iPSCs are then washed with DPBS and incubated with isolated cytotoxic T cells for 24 hours. Cytotoxic T cells are isolated from PBMCs using the EasySep™ hCD8+ T Cell Isolation Kit (StemCell Technologies). T cell activation is measured by ELISpot assays via detection of secreted INF-γ and Granzyme B. Plates are scanned with the ELISpot Analyzer (ImmunoSpot) and INF-γ and Granzyme-B specific cytokine release events are quantified. The number of Granzyme B and IFN-γ spots is expected to be lower in wells where cytotoxic T cells were incubated with TAPBP-KO, TAPBP/TAPBPL Double-KO and β2M-KO, when compared with unmodified, tapasin-expressing iPSCs.
NK cells: NM cells are isolated from fresh blood using the EasySep™ Direct hNK Cell Isolation Kit (StemCell Technologies) and expanded for 14 days using NK MACS Medium (Miltenyi Biotec) plus IL-2. Expanded NK cells are co-cultured with TAPBP-KO, TAPBP/TAPBPL Double-KO, β2M-KO, and unmodified iPSCs previously treated with IFN-γ. NK cells are recovered from the co-culture, washed, and flow cytometry ise performed to evaluate the expression of activation markers, such as intracellular IFN-γ and CD107a. B2M-KO iPSCs are expected to induce a higher NK cell activation (as percentage of IFN-γ⁽⁺⁾/CD107a⁽⁺⁾ cells) profile is expected to be higher when NK cells were co-cultured with NK cells activation markers are expected to cells are expected to express higher

### Example 4

### Immunogenicity and Survival of Tapasin Null- and Tapasin/Tapasin-Like-Null Engineered Cells in Humanized Mouse Models of Immune Cell Function In Vivo

TAPBP-KO (Tapbp-null), TAPBP/TAPBPL Double-KO (Tapbp/Tapbpl-null), B2M-KO and unmodified iPSCs are tested for half-life in circulation and immunogenicity following infusion into the circulation of ‘humanized’ SCID mice harboring the full array of human leukocytes reconstituted from human CD34+ hematopoetic stem cells. These experiments are performed in humanized mice derived from CD34+ cells or peripheral blood mononuclear cells of non-HLA-matched individuals relative to the parental iPSC line used to produce the Tapbp-null and Tapbp/Tapbpl-null iPSCs. Tapbp-null and Tapbp/Tapbpl-null iPSCs are detected and quantified on the basis of expression of a fluorescent protein, luciferase enzyme, or other traceable marker. Tapbp-null and Tapbp/Tapbpl-null iPSCs are expected to exhibit reduced induction of T cell and NK cell cytokines involved in systemic inflammatory response syndrome (SIRS) and exhibit greater half-lives of survival in circulation in vivo, as compared with wild type iPSCs, and this will be particularly evident in non-HLA-matched humanized mice recipients.

In similar experiments, Tapbp-null and Tapbp/Tapbpl-null iPSCs and the control parental wild type iPSCs expressing Tapbp and Tapbpl, along with a variety of iPSC-derived mature cells types that may include fibroblasts, hepatocytes, and cardiomyocytes, are tested for immunogenicity by xenotransplantation in accessible anatomic locations, such as under the renal capsule, of humanized mice reconstituted from CD34 cells of HLA-matched and non-HLA-matched individuals. Tapbp-null and Tapbp/Tapbpl-null iPSCs are expected to generate teratomas in greater number and size after xenotransplantation in accessible anatomic locations, such as under the renal capsule, and display less infiltration by inflammatory immune cells after recovery and post-mortem examination, as compared with control parental wild type iPSCs. Similarly, greater survival and less inflammatory cell infiltration should be observed when different Tapbp-null and Tapbp/Tapbpl-null iPSC-derived mature cells types are compared with wild type mature cell types derived from parental control wild type iPSCs.

1. A method for producing a cell which exhibits reduced antigen presentation by the major histocompatibility complex type I (MHC-I), said method comprising decreasing or eliminating activity of a tapasin protein or homolog thereof in one or more cells to be transplanted into an allogeneic recipient in need thereof so that a cell which exhibits reduced antigen presentation by MHC-I is produced.

2. A method for transplanting one or more cells into an allogenic recipient in need thereof, said method comprising:
(a) preparing one or more cells in which activity of an endogenous tapasin protein or homolog thereof is decreased or eliminated in accordance with the method of claim 1; and
(b) transplanting the one or more cells into the allogeneic recipient in need thereof.

3. The method of claim 1, wherein activity of the tapasin protein or homolog thereof is decreased or eliminated by inhibiting expression of a nucleic acid sequence encoding the tapasin protein or homolog thereof.

4. The method of claim 3, wherein expression of the nucleic acid sequence encoding the tapasin protein or homolog thereof is inhibited using a gene knockout system or a gene editing system, by modifying the one or more cells to express a nucleic acid sequence encoding a dominant-negative mutant of tapasin or the homolog thereof, or by modifying the one or more cells to constitutively express a viral immunoevasin protein.

5. (canceled)

6. The method of, claim 4 wherein the gene editing system comprises a CRISPR-Cas system, a zinc finger nuclease (ZFNs) system, or a transcription activator-like effector-based nucleases (TALEN) system.

7-8. (canceled)

9. The method of, claim 4 wherein the immunoevasin protein is a human cytomegalovirus (HCMV) US3 protein, a HCMV US6 protein, an adenovirus E3-19K protein, a pseudorabies US3 protein, a pseudorabies UL49.5 protein, a herpes simplex virus ICP47 protein, an equine herpesvirus (EHV) UL49.5 protein, a bovine herpesvirus (BHV) UL49.5 protein, an Epstein-Barr virus (EBV) BFNL2a protein, a cowpox virus (CPV) CPXV12 protein, or any combination thereof.

10. The method of claim 1 further comprising modifying the one more cells to introduce one or more bacteriophage integrase acceptor sites into the cellular genome.

11. The method of claim 1, wherein the one or more cells are embryonic stem cells (ESCs), induced pluripotent stem cells (iPSCs), hematopoietic stem cells (HSCs), mesenchymal stem cells (MSCs), natural killer cells, natural killer cells expressing a chimeric antigen receptor (CAR-NK cells), T lymphocytes, T lymphocytes expressing a chimeric antigen receptor (CAR-T cells), hematopoietic cell types, and cell types intended for use in regenerative medicine applications including skeletal muscle cells, smooth muscle cells, gastric/intestinal/colonic epithelial cells, neuroendocrine cells, endothelial cells, cardiomyocytes, hepatocytes, pancreatic islet cells, neurons, glial cells, skin cells, chondrocytes, osteoblasts, or retinal pigment epithelium cells.

12. (canceled)

13. The method of claim 1 wherein the homolog of tapasin is TAP Binding Protein-Like (TAPBPL).

14. A cell which exhibits reduced antigen presentation by the major histocompatibility complex type I (MHC-I), said cell comprising a cellular genome which lacks at least a portion of an endogenous tapasin gene or homolog thereof such that the expression of the tapasin protein or the homolog thereof in the cell is decreased or eliminated or which is modified to express a nucleic acid sequence encoding a dominant-negative mutant of tapasin or a homolog thereof and/or a nucleic acid sequence encoding viral immunoevasin protein.

15. (canceled)

16. The cell of, claim 14 wherein the immunoevasin protein is a human cytomegalovirus (HCMV) US3 protein, a HCMV US6 protein, an adenovirus E3-19K protein, a pseudorabies US3 protein, a pseudorabies UL49.5 protein, a herpes simplex virus ICP47 protein, an equine herpesvirus (EHV) UL49.5 protein, a bovine herpesvirus (BHV) UL49.5 protein, an Epstein-Barr virus (EBV) BNFL2a protein, a cowpox virus (CPV) CPXV12 protein or any combination thereof.

17. The cell of claim 14 which is an embryonic stem cells (ESCs), induced pluripotent stem cells (iPSCs), hematopoietic stem cells (HSCs), mesenchymal stem cells (MSCs), natural killer cells, natural killer cells expressing a chimeric antigen receptor (CAR-NK cells), T lymphocytes, T lymphocytes expressing a chimeric antigen receptor (CAR-T cells), hematopoietic cell types, and cell types intended for use in regenerative medicine applications including skeletal muscle cells, smooth muscle cells, gastric/intestinal/colonic epithelial cells, neuroendocrine cells, endothelial cells, cardiomyocytes, hepatocytes, pancreatic islet cells, neurons, glial cells, skin cells, chondrocytes, osteoblasts, or retinal pigment epithelium cells.

18. (canceled)

19. The cell of claim 14 wherein the homolog of tapasin is TAP Binding Protein-Like (TAPBPL).

20. A nucleic acid sequence comprising a 5′ homology arm complementary to a tapasin gene or portion thereof or homolog thereof, a 3′ homology arm complementary to a tapasin gene or portion thereof or homolog thereof, and at least one exogenous polynucleotide.

21. The nucleic acid sequence of claim 20 wherein the 5′ and 3′ homology arms are nucleic acid fragments with complementary sequences to an adjacent chromosomal transfer site that is present within in human chromosome 6 (NCBI Reference Sequence (NC_000006.12).

22. The nucleic acid sequence of claim 20 wherein the 5′ homology arm and the 3′ homology arm are complementary to different parts of a tapasin gene region of a host cell.

23. The nucleic acid sequence of claim 20 further comprising a portion of an exon region of the tapasin gene or the homolog thereof.

24. The nucleic acid sequence of claim 20 wherein the 5′ and/or 3′ homology arms exhibit at least 60% sequence identity with a nucleic acid sequence encoding tapasin as set forth in an amino acid sequence comprising SEQ ID NO:14, 15, 16, 17, 18, 19, 20, 21, 22 or 23 or portion thereof or homolog thereof.

25. The nucleic acid sequence of claim 24 wherein the homolog of tapasin is TAP Binding Protein-Like (TAPBPL) as set forth in SEQ ID NO:24 or 25 or portion thereof.

26. The nucleic acid sequence of claim 20 wherein the exogenous polynucleotide comprises a site-specific recombinant sequence recognized by a site-specific recombinase.

27. The nucleic acid sequence of claim 20 wherein the exogenous polynucleotide encodes a chimeric antigen receptor.

28. A vector comprising the nucleic acid sequence of claim 20.

29. A host cell comprising the nucleic acid sequence of claim 20.

30. A method for inhibiting expression or function of tapasin or a homolog thereof in a host cell, said method comprising incorporation the nucleic acid sequence of claim 20 into the host cell.
